(19) United States
(12) Patent Application Publication
(10) Pub. No.: US 2015/0025034 A1
(43) Pub. Date: Jan. 22, 2015

(54) COMPOSITION FOR CARE AND HYGIENE OF THE AUDITORY CANAL

(71) Applicant: LABORATOIRES GILBERT, HEROUVILLE SAINT CLAIR (FR)

(72) Inventor: Laurent Batteur, Cambes En Plaine (FR)

(21) Appl. No.: 14/383,688

(22) PCT Filed: Mar. 7, 2013
(86) PCT No.: PCT/EP2013/054560

§ 371 Date: Sep. 8, 2014

(30) Foreign Application Priority Data
Mar. 7, 2012 (FR) 1252048

(51) Int. Cl. A61K 8/60; A61Q 19/00; A61K 8/362; A61K 8/41; A61K 8/42; A61K 8/34

(52) U.S. Cl. CPC A61K 8/602; A61K 8/42; A61K 8/345; A61K 8/362; A61K 8/41; A61Q 19/00; A61K 2800/70
USPC 514/53

(57) ABSTRACT

The aim of the invention is to propose a composition for the care and hygiene of the auditory canal, in particular a cerumenolytic composition, which is effective for dissolving and preventing the appearance of plugs of cerumen, which is free from products irritating to the auditory canal or a source of allergy, such as preservatives or surfactants of animal origin, which is biodegradable and non-toxic for the environment, which can be used under non-sterile conditions while ensuring satisfactory preservation and stability and which does not require rinsing after use thereof. To this end, the invention concerns a composition for the care and hygiene of the auditory canal that is characterised in that it comprises a pair of surfactants consisting of an N-acyl sarcosinate sodium salt having an HLB greater than or equal to 25 and a sucrose fatty acid ester having an HLB greater than or equal to 11.

The present invention concerns a composition for the care and hygiene of the auditory canal, in particular intended to facilitate the elimination of residues of cerumen and to prevent the formation of plugs of cerumen in the auditory canal.

Cerumen is a substance secreted by the sebaceous glands in the outer auditory canal, in which if fulfils a role of barrier to bacteria and fungi as well as a lubrication role.

In some individuals, depending on their age and their genetic makeup, or when the ears are too highly stressed, cerumen is secreted in excess and in the long term produces a blockage.

Effective compositions for the care and hygiene of the ear based on surfactant compounds of animal origin are known for example. By way of example, one known composition comprises, as surfactants, TEA-cocoyl hydrolysed collagen, betaine and a modified polyethylene glycol. The drawback of such a formulation, apart from the fact that it contains a surfactant compound of animal origin, lies in the need for rinsing of the auditory canal after application of the composition. This is because the formulation is aggressive and irritant to the skin, because of the chemical structure thereof. Furthermore, this formulation comprises preservatives of the paraben type, which it would also be necessary to be rid of.

The aim of the invention is to propose a composition for the care and hygiene of the auditory canal, in particular a cerumenolytic composition, which is effective for dissolving and preventing the appearance of plugs of cerumen, which is free from products irritating to the auditory canal or a source of allergy, such as preservatives or surfactants of animal origin, which is biodegradable and non-toxic for the environment, which can be used under non-sterile conditions while ensuring satisfactory preservation and stability, and which does not require rinsing after use thereof.

To this end, the invention concerns a composition for the care and hygiene of the auditory canal that is characterised in that it comprises a pair of surfactants consisting of an N-acyl sarcosinate sodium salt having an HLB greater than or equal to 25 and a sucrose fatty acid ester having an HLB greater than or equal to 11.

Preferentially, the composition contains between 4% and 9%, by weight with respect to the total weight of the composition, of the pair of aforementioned surfactants.

The pair of surfactants consists of ionic and non-ionic surfactants both of plant origin. The nature of the surfactants chosen eliminates the need for rinsing of the auditory canal after application of the composition since they are both very well tolerated and non-irritant to the skin. They have a sufficiently high molecular mass not to cross the cell membrane and neither absorb nor denature keratins. They are therefore without toxic risks. In addition, they are of plant origin and biodegradable.

The chemical formula of acyl sarcosinate sodium salts is as follows (I):

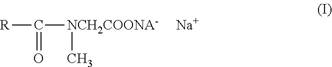

in which R is a fatty acid with a chain length of C10 to C18.

Acyl sarcosinates are characterised by an alkylamide group with a hydrophobic part derived from the linear part of the fatty acid. The hydrophilic part is formed by the sarcosine.

Acyl sarcosinates are anionic surfactants. Such surfactants are generally known to be irritant to the skin. However, the applicant company has nevertheless selected acyl sarcosinates since they have both an effective detergent action because of the high HLB thereof but also an absence of irritation of the skin. The hydrophilic-lypophilic balance (HLB), which makes it possible to specify the rather hydrophilic or rather lypophilic nature of a surfactant, is normally used as one of the criteria of choice of a surfactant. This HLB parameter, which increases with the hydrophilic character of the surfactant, was defined by Griffin.

Preferentially, the N-acyl sarcosinate sodium salt according to the invention comprises a linear chain derived from a fatty acid comprising 10 to 14 carbon atoms. The length of the chain participates in the detergent function of the surfactant. The longer the chain, the less detergent is the N-acyl sarcosinate.

The hydrophobic and hydrophilic poles of acyl sarcosinates are compatible with keratin through their similar structure and through the absence of sulphated groups, which are replaced with a carboxylic group. Acyl sarcosinates are considered to be true “interrupted soaps”. This is because the N-methylamide group is placed between the aliphatic chain and the carboxylic group of a “soap” structure. The acid-base equilibrium of the sarcosinates confers on them maximum dermatological compatibility.

Their special feature is to couple with protein substrates with a pH of between 4 and 7, where the N-acyl sarcosine group is blocked by bonds that easily form with the keratin structures. Acyl sarcosinates are also excellent foam boosters and are capable of giving a stable foam compared with other foam boosters.

This surfactant therefore acts in a detergent fashion on the lypophilic components of cerumen and thus fragments it before elimination.

It should be noted that the term detergent is often employed improperly to designate surfactant compounds. By definition, a detergent has a cleaning capability and it is therefore a surfactant compound. On the other hand, a surfactant compound may not have detergent properties.

Sucrose fatty acid esters are non-ionic surfactants consisting of a hydrophilic group, sucrose, and a lypophilic group, the fatty acid. Since sucrose comprises eight hydroxyl functions, it is possible to produce a range of esters from the sucrose monoester up to the sucrose octaester. They are non-toxic and non-irritant and have excellent biodegradability.

Sucrose fatty acid esters suitable in the context of the invention are of plant origin. In addition they have an HLB greater than 11. They thus have an effective wetting property that acts on the cerumen and thus facilitates detachment thereof and clearing of the auditory channel. By way of example, sucrose stearate or sucrose laurate can be cited.

The N-acyl sarcosinate sodium salt having an HLB greater than or equal to 25 and the sucrose fatty acid ester having an HLB greater than 11 chosen are complementary in their actions. This is because the N-acyl sarcosinate sodium salt provides mainly the detergent activity of the composition and the sucrose fatty acid ester for its part provides the wetting power. The result is, during the use of the composition, lysis of the cerumen and detachment and then elimination thereof, without any irritation reaction.

The composition according to the invention does not comprise any other detergent active principles apart from the aforementioned pair, nor any preservative. The choice of the detergent pair confers great stability on the composition and great efficacy and makes it possible to dispense with the addition of a preservative. Furthermore, use thereof does not require any rinsing of the auditory channel following the application of the composition. The composition does not comprise any active principles other than the aforementioned pair of surfactants.

Thus, advantageously, the composition comprises between 3% and 6%, preferentially between 4% and 5%, more preferentially 4.5%, by weight with respect to the weight of the composition, of N-acyl sarcosinate sodium salt having an HLB greater than or equal to 25, and between 1% and 3%, preferentially between 1.8% and 2.2%, more preferentially 2%, by weight with respect to the weight of the composition, of sucrose fatty acid ester having an HLB greater than or equal to 11.

The choice of these quantities, and the ratio between the two quantities, makes it possible to observe the complementarity of action of the surfactant compounds.

Unless indicated to the contrary, the percentages expressed in the present application are by weight with respect to the total weight of the composition.

According to a preferred embodiment of the invention, the N-acyl sarcosinate sodium salt is sodium lauroyl sarcosinate. This compound has made it possible to obtain excellent results since it proves to be particularly gentle, non-irritant and effective in terms of detergent activity. The HLB thereof is approximately 29, which guarantees satisfactory detergent activity.

According to an equally preferred embodiment of the invention, the sucrose fatty acid ester has an HLB of between 12 and 15. Preferentially again, it is sucrose laurate, for which very good results have been obtained, in particular in combination with sodium lauroyl sarcosinate. Sucrose laurate has in fact very good wetting efficacy compared with the other sucrose fatty acid esters.

Adjuvants and additives are also provided in the composition.

For example, the composition advantageously comprises a moistening agent, in particular methylpropanediol. The moistening agent reinforces the anti-adherent effect of the composition, thus facilitating the detachment of the cerumen in the auditory canal. This is especially advantageous in the context of a so-called dry cerumen, composed mainly of keratinised substances that adhere to the auditory canal. Methylpropanediol, which is a compound of natural origin, also has the advantage of producing an effect on the microbial protection of the composition.

The composition comprises preferentially between 4.5% and 6%, more preferentially 5%, of moistening agent.

Other agents may also be incorporated in the composition according to the invention, such as ethylhexylglycerine, capryloyl glycine and/or tetrasodium glutamate diacetate. Capryloyl glycine is an amphiphilic molecule but is not a detergent. Advantageously, the composition comprises the combination of these three compounds. The applicant company has in fact observed that the combination of these three substances makes it possible to obtain a synergic effect on the protection of the composition vis-à-vis microbial contamination.

In order to ensure perfect compatibility with cerumen and the auditory canal, an acid compound, such as citric acid, can be used to regulate the pH to a target of 5.75 or between 5 and 6. According to one embodiment of the invention, the composition is in the form of a single dose or a spray.

Finally, the composition comprises an aqueous solvent such as water.

A preferred composition according to the invention will comprise a pair of surfactants consisting of sodium lauroyl sarcosinate and sucrose laurate; by way of additives, ethylhexylglycerine, capryloyl glycine and tetrasodium glutamate diacetate; by way of moistening agent, methylpropanediol, and by way of aqueous solvent, water.

Advantageously, the composition is in the form of a spray or a single dose.

The invention also concerns a composition as described previously for use thereof in the cleaning and care of the auditory channel, for eliminating residues of cerumen and preventing the formation of plugs of cerumen in the auditory canal.

The invention will be better understood from a reading of the following example embodiments, which are supposed to be illustrative and non-limitative.

### Example 1

### Cerumenolytic Composition CE1 (Table 1

TABLE 1

| CASE NO. | NAME | % BY WEIGHT | ROLE |
|---|---|---|---|
| 7732-18-5 | Aqueous phase | 86.90 | Solvent/excipient |
| | (including water | | |
| | 86.4%) | | |
| 504-63-2/ | Propanediol | 5% | Moistening agent |
| 26264-14-2 | | | |
| 137-16-6 | Sodium lauroyl | 4.50 | Anionic surfactant |
| | sarcosinate¹ | | |
| 25339-99-5 | Sucrose laurate² | 2.00 | Anionic surfactant |
| 14246-53-8 | Capryloyl glycine | 0.60 | Additive |
| 70445-33-9 | Ethylhexylglycerin, | 0.4995 | Additive |
| 77-92-9/ | Citric acid | 0.30 | Additive |
| 5949-29-1 | | | |
| 51981-21-6 | Tetrasodium | 0.20 | Additive |
| | glutamate diacetate | | |
| TOTAL | | 100.000 | |

¹Sodium lauroyl sarcosinate in solution at 30% active material; PROTELAN LS 9011 (Z&S) or PERLASTAN L-30 HFC (Ballu)
²Sucrose laurate in solution at approximately 40% diluted in alcohol; SISTERNA LC70-C

Study of Stability, Surface Tension and Rheology of Composition CE1:

Microemulsions were produced by mixing composition CE1 (7.987 g) in squalene (3.008 g) and water (18.128 g); squalene is a model simulating the sebum secreted in the auditory channel. The emulsion is left at ambient temperature for 24 hours. No demixing is observed; the emulsions are stable.

The mean size of the droplets of emulsions, measured by means of the Zetasizer nanoZS apparatus from Malvern Instruments, using the non-invasive light backscatter method (sensitivity of 0.6 nanometres to 6 micrometres) is around 198.9 nm. This data confirms the stability of the composition and the efficacy of the pair of surfactants. This is because, the smaller the size of the droplets of a microemulsion, the greater the stability of the microemulsion. Moreover, the smaller the size of the droplets, the more effective is the surfactant and capable of forming oil droplets.

The surface tension measured by means of the K100 tensiometer (Krüss) according to the Wilhelmy plate method is 25.841 mN/m. This measurement proves good wettability of the composition in squalene. The rheology measured by means of the Physica MCR 301 flowmeter (Anton Paar) is also satisfactory.

In Vitro Assessment of the Cerumenolytic Effect of the Composition According to the Invention on Plugs of Human Cerumen:

The activity was assessed through the loss of weight of the plugs of cerumen caused by the dissolution of part of this plug, or more precisely the loss was calculated between the initial dry weight and the remaining dry weight after action of the product in the test.

Reference product: an aerosol spray based on pure sea water (nitrogen propellant), hereinafter referred to as T1, an aerosol spray containing sodium bicarbonate and polysorbate as active principles, hereinafter referred to as T2, and ultrapure water (type 1, Milli-Q water) are used as references. T1 and T2 are commercially available compositions used by way of compositions for the care and hygiene of the auditory channel and judged effective.

12 plugs of human cerumen, to a total weight of 1.55 grams, came from patients from the Ear, Nose and Throat Department of the Saint-Grégoire Hospital (35760). The plugs were mixed in order to obtain a homogeneous plug. Samples of 30 mg (+/−2 mg) came from the homogeneous plug and were placed in previously weighed glass tubes.

Compositions T1 and T2, ultrapure water and CE1 were applied separately at the rate of 1 ml per plug of cerumen. The incubation time of the compositions on the plugs of cerumen was 1 hour in the presence or not of the CE1 composition, the reference compositions T1, T2 and ultrapure water. The incubation was carried out at 37° C. in a water bath. The tubes were agitated at 500 rpm on a plate agitator every 10 minutes for 1 minute. At the end of one hour, the tubes were weighed.

The effect sought is the dissolution of the plug. This dissolution is assessed by the loss of weight of the plug between the initial dry weight and the dry weight after incubation.

Weighing of the Cerumen:

After incubation, the tubes were centrifuged at 1000 rev/min (rise to the speed and then stoppage of the centrifugation), and the supernatant was taken off and weighed after transfer into a new tube (previously weighed). The tube containing the residue of cerumen was also weighed. The tubes were transferred into an oven at 90° C. for 48 hours in order to evaporate the compositions, and then the tubes were weighed.

Weighing of the Dry Residues:

1 ml of composition CE1 and the reference compositions were added separately in previously weighed tubes and put in an oven at 90° C. for 48 hours in order to determine the quantity of dry residues of each composition. This step was carried out in triplicate.

Results:

Expression of the Results:
Mean weight of lysate in mg and as a percentage of lysis with respect to the weight before incubation.
Statistical tests carried out on the lysate values.
Test on the difference between the averages of the various groups by analysing the variance at a factor (ANOVA 1; α<0.05). In the event of significance of the variance analysis, comparison of the average between each “treated” group (Dunnett test; α<0.05).

Observations:

The variances are homogeneous (p0.734). The Anova 1 test has a significance of p<0.01, showing that there exists a difference between the products, allowing comparison of the averages between the products tested.

A loss of weight of 20.7% of the cerumen alone was observed.

The pure water caused a dissolution of 51.5% of the human cerumens after 1 hour of incubation.

The reference composition T1 caused a dissolution of 43.6% of the human cerumens after 1 hour of incubation.

The reference composition T2 caused a dissolution of 64.8% of the human cerumens after 1 hour of incubation.

The composition according to the invention CE1 caused a dissolution of 61% of the human cerumens after 1 hour of incubation.

Thus the composition according to the invention CE1 caused significantly the dissolution of human cerumen compared with ultrapure water (p<0.001), cerumen alone (p<0.001), and composition T1 (p<0.001). There is no significant difference between CE1 and the reference composition T2 (p>0.05),

In conclusion, this test proves that the composition according to the invention is effective, or even that it has efficacy superior to some products of the prior art themselves judged to be effective. The use of the pair of surfactants of plant origin and the choice of the quantity thereof makes it possible to formulate a cerumenolytic composition based on gentle compounds, not requiring any rinsing after application and without danger for the environment.

Assessment of the Tolerance of a Composition According to the Invention:

A comparative test between composition CE1 and a composition comprising a surfactant of animal origin that is TEA-cocoyl hydrolysed collagen (8%), and coco betaine (1.8%), PEG-120 methyl glucose dioleate (1.5%) and preservatives (methylparaben, phenoxyethanol, butylparaben, ethylparaben and isobutylparaben) was carried out on an animal model. In a condition of repeated use over 6 days on a rinsed and non-rinsed area, the composition comprising a surfactant of animal origin is more irritating than composition CE1.

Clinical Test:

A study under the control of specialist ear, nose and throat doctors was carried out on the composition CE1.

The volunteers were 19 men and 40 women, aged 48+/−12 years, preferably having a sensation of hypersecretion of cerumen causing an auditory problem or complaining of a sensation of blocked ear after a shower or shampooing. They could be wearers of auditory prostheses.

During the 14 days preceding the study, the volunteers should no longer clean their ears. They should not clean their ears throughout the period of the study, that is to say 21 days, other than with the test product only in the ear that was indicated to them, at the rate of 2 uses per week.

The following results were observed by four ear, nose and throat specialists:
a significant difference concerning the quantity of cerumen observed in the treated ears as against untreated reference ears. The product was efficacious on the quantity of cerumen observed;
a significant difference concerning the quality of the cerumen observed in the treated ears as against untreated reference ears. The product was efficacious on the softening of cerumen.

Furthermore, the following efficacy criteria were quantified by the ear, nose and throat specialists:
softening of the cerumen observed in 96% of the persons,
regulation of the production of cerumen observed in 94% of the persons;
prevention of the appearance of cerumen observed in 90% of the persons.

The comments of the volunteers subjected to a test treatment with the composition CE1 are set out below:
gives gentle cleaning: yes for 86% of the volunteers,
facilitates elimination of excess cerumen: yes for 82%,
prevents the appearance of cerumen: yes for 77%,
regulates the production of cerumen: yes for 77%.

1. Composition for care and hygiene of the auditory canal, the composition comprising a pair of surfactants consisting of an N-acyl sarcosinate sodium salt having an HLB greater than or equal to 25 and a sucrose fatty acid ester having an HLB greater than or equal to 11.

2. Composition according to claim 1, wherein the composition comprises between 3% and 6%, preferentially between 4% and 5%, more preferentially 4.5%, by weight with respect to the weight of the composition, of said N-acyl sarcosinate sodium salt and between 1% and 3%, preferentially between 1.8% and 2.2%, more preferentially 2%, by weight with respect to the weight of the composition, of said sucrose fatty acid ester.

3. Composition according to claim 1, wherein said N-acyl sarcosinate sodium salt is sodium lauroyl sarcosinate.

4. Composition according to claim 1, wherein said sucrose fatty acid ester is sucrose laurate.

5. Composition according to claim 1, wherein it further comprises at least one aqueous solvent, at least one moistening agent and/or at least one additive.

6. Composition according to claim 5, wherein the moistening agent is methylpropanediol.

7. Composition according to claim 5, wherein it comprises, by way of additives, ethylhexylglycerine, capryloyl glycine and tetrasodium glutamate diacetate.

8. Composition according to claim 1, wherein the composition is in the form of a single dose or a spray.

9. Composition according to claim 1, wherein it comprises:
a pair of surfactants consisting of sodium lauroyl sarcosinate and sucrose laurate,
by way of additives, ethylhexylglycerine, capryloyl glycine and tetrasodium glutamate diacetate,
by way of moistening agent, methylpropanediol,
by way of aqueous solvent, water.

10. Composition as described in claim 1, for use thereof in the cleaning and care of the auditory canal, for eliminating residues of cerumen and preventing the formation of plugs of cerumen in the auditory canal.
